(19) United States
(12) Patent Application Publication
(10) Pub. No.: US 2025/0299506 A1
(43) Pub. Date: Sep. 25, 2025

(54) CLUSTER-BASED HISTOPATHOLOGY PHENOTYPE REPRESENTATION LEARNING BY SELF-SUPERVISED MULTI-CLASS TOKEN HIERARCHICAL VISION TRANSFORMER

(71) Applicant: Ventana Medical Systems, Inc., Tucson, AZ (US)

(72) Inventors: Mohammad Saleh MIRI, San Jose, CA (US); Shivam KALRA, Ontario (CA); Jiarong YE, State College, PA (US)

(73) Assignee: Ventana Medical Systems, Inc., Tucson, AZ (US)

(21) Appl. No.: 19/229,969

(22) Filed: Jun. 5, 2025

Related U.S. Application Data
(63) Continuation of application No. PCT/US2023/083003, filed on Dec. 7, 2023.
(60) Provisional application No. 63/386,617, filed on Dec. 8, 2022.

(51) Int. Cl. G06V 20/69; G01N 33/483; G06T 7/00; G06V 10/762; G06V 10/764; G16H 50/20

(52) U.S. Cl. CPC G06V 20/698; G01N 33/4833; G06T 7/0012; G06V 10/762; G06V 10/764; G06V 20/695; G16H 50/20; G06T 2207/20081; G06T 2207/20084; G06T 2207/30024; G06V 2201/03

(57) ABSTRACT

The system and method for processing a digital pathology image using a machine learning model that includes a self-supervised hierarchical Vision Transformer (ViT) configured to perform unsupervised clustering with multiple classification tokens. The method includes receiving a digital pathology image that depicts a tissue slice stained with histological dyes. The digital pathology image may be processed to generate a result comprising multiple predicted classifications of individual patches of the digital pathology image. The result is generated by a machine-learning model using a self-supervised hierarchical Vision Transformer (ViT) that may further comprise a multi-head self-attention module configured to predict a crosspatch relevance metric using an attention mechanism for each individual patch in the digital pathology image thereby assigning the individual patches to a cluster based on the crosspatch relevance metrics.

### CROSS-REFERENCE TO RELATED APPLICATIONS

This application is a continuation of International Patent Application No. PCT/US2023/083003, filed on Dec. 7, 2023, which claims the benefit of and the priority to U.S. Provisional Application No. 63/386,617, filed on Dec. 8, 2022. The entire disclosures of the aforementioned applications are incorporated by reference herein in their entireties for all purposes.

### BACKGROUND

Access to large-scale and high-quality datasets may prove a primary driver in machine learning. For example, ImageNet is a data set that had been used to train computer-vision models that perform remarkably well when processing natural images. Meanwhile, for medical image analysis tasks, labelled data may be scarce and expensive since annotations from multiple experts may be often required and crowdsourcing may not be an option. Furthermore, inter-observer variability among medical experts may affect the quality of the dataset. Accordingly, it may be frequently both cost and time prohibitive to assemble a large and high-quality dataset for medical imaging analysis tasks, which may limit the progress of research and model development in this field.

Unsupervised machine learning is an approach that trains a model using unlabeled data. Unsupervised learning could provide a solution to the above-mentioned challenges and promote the development of more accurate artificial intelligence (AI) models. Transfer learning is a technique where a model may be pre-trained (e.g., using the ImageNet dataset) and then fine-tuned using a type of data of interest (e.g., medical images). This method is advantageous because the ImageNet dataset is typically much larger than a medical dataset thereby providing a model with good foundation to understand fundamental image features. Nevertheless, challenges arise due to potential disparities in features and patterns between natural-scene images from ImageNet and medical images, impeding the model's convergence and potentially extending the training process.

Histopathology has seen widespread adoption of digitization, offering unique opportunities to increase objectivity and accuracy of diagnostic interpretations through machine learning. Digital images of tissue specimens may exhibit significant complexity and heterogeneity from the preparation, fixation, and staining protocols, among other factors. This variety further may exacerbate the accessibility to a large-labelled dataset in digital pathology as compared with any other medical imaging modalities. Furthermore, each tissue specimen image is generally a gigapixel file which may require significantly more labeling effort from an expert leading to higher inter/intra-observer variability and mis-localization of lesions. These challenges may strengthen the imperative of utilization of unsupervised machine learning approaches to leverage vast amounts of unlabeled data in digital pathology domain.

### SUMMARY

Some embodiments of the present disclosure relate to processing a digital pathology image using a machine learning model that includes a self-supervised hierarchical Vision Transformer (ViT) configured to perform unsupervised clustering. The computer-implemented method includes accepting digital pathology images depicting tissue slice stained with histological dyes (e.g., without any accompanied pathologists' annotations). The digital pathology images are processed to generate result comprising multiple predicted classifications of individual patches of these images (e.g., in an unsupervised manner). The result is generated by a machine-learning model using a self-supervised hierarchical ViT as a backbone encoder to capture semantically meaningful fine-grained regions of interest detailed to the pixel level. The hierarchical ViT further includes a multi-head self-attention module configured to predict a crosspatch relevance metric using an attention mechanism for each individual patch in a digital pathology image. Based on the crosspatch relevance metrics, the individual patches may be assigned to a cluster.

The multiple predicted classification tokens may indicate, predict or correspond to one or more of: a type of tissue, a magnification level, a diagnostic category (e.g., a nondiagnostic category, negative for malignancy category, atypical category, neoplastic: benign category, suspicious category, or positive for malignancy category) characterizing a histological feature (e.g., cytological feature), or a prediction as to whether the digital pathology image depicts a particular histological feature of malignancy or an extent to which the digital pathology image depicts the particular histological feature of malignancy. The particular histological feature may include high cellularity, cellular enlargement, cellular discohesiveness, a high nuclear-to-cytoplasm ratio, nuclear hyperchromasia, prominent nucleoli, large nucleoli, abnormal nuclear-chromatin distribution, high mitotic activity, abnormal nuclear membrane, cellular pleomorphism, nuclear pleomorphism, or tumor diathesis.

The multiple predicted classification tokens may characterize what is being depicted in a portion or all of the digital pathology image. For example, the portion may include a patch or a pixel.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product tangibly embodied in a non-transitory machine-readable storage medium, including instructions configured to cause one or more data processors to perform part or all of one or more methods or processes disclosed herein.

In some embodiments, a system is provided that includes one or more means to perform part or all of one or more methods or processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates an exemplary workflow of a self-supervised learning (SSL) framework.

FIG. 2A shows an illustrative example of a self-supervised contrastive method, DINO (Distillation of Information via Non-parametric contrasting).

FIG. 2B shows an illustrative example of a vision transformer from the FIG. 2A.

FIG. 3 illustrates an example process of patching and embedding from the FIG. 2A.

FIG. 4 depicts an exemplary implementation of a backbone transformer encoder cluster-based histopathology phenotype representation learning by self-supervised multi-class-token hierarchical vision transformer (Cypher ViT) in accordance with some embodiment of the present disclosure.

FIG. 5 illustrates an example architecture of a Cypher ViT attention module from the FIG. 4.

FIG. 6 shows an example working of attention mechanism in accordance with an embodiment of the present disclosure.

FIG. 7 shows an example flowchart of a computer-implemented method to process a digital pathology image using a machine-learning model in accordance with some embodiments of the present disclosure.

FIG. 8A illustrates two-dimensional (2D) UMAP (Uniform Manifold Approximation and Projection) visualization of feature embeddings extracted from an example implementation of the state-of the-art self-supervised learning (SSL) frameworks pre-trained on a dataset.

FIG. 8B illustrates 2D UMAP visualization of feature embeddings extracted from an example implementation of a state-of-the-art SSL framework iBOT (image BERT training with Online Tokenizer) and the present disclosure pre-trained on a dataset.

FIG. 9 shows retrieval results from a set of query images in accordance with an example implementation of the present disclosure.

FIG. 10A shows attention maps of multiple predicted classification tokens extracted from a learnable multi-class tokens at a final stage of an example implementation of the present disclosure.

FIG. 10B shows attention maps of multiple predicted classification tokens extracted from a learnable multi-class tokens at a final stage of an example implementation of the present disclosure.

### DETAILED DESCRIPTION

Some embodiments of the present disclosure relate to processing digital pathology images using a machine learning model that includes a self-supervised hierarchical Vision Transformer (ViT) configured to perform unsupervised clustering with multiple classification tokens. More specifically, the machine-learning model can be configured with multiple levels, each level assembles semantically similar patches into fixed number of classification tokens. The classification tokens can be used by an attention mechanism to determine how similar various patches are. For example, classification labels may be used to predict how relevant a given patch is to each of one or more other patches, which can then be used to support assigning individual patches to a cluster. The classification tokens are learned in a self-supervised manner and/or may relate to histological semantics.

In some embodiments, a framework is provided for Cluster-based histopathology Phenotype Representation learning by self-supervised multi-class-token hierarchical ViT (Cypher ViT) as a novel backbone encoder to replace the regular ViT (Dosovitskiy et al. “An image is worth 16×16 words: Transformers for image recognition at scale.” arXiv preprint arXiv: 2010.11929, 2020, which is hereby incorporated by reference in its entirety for all purposes) in an SSL pipeline. In some embodiments, the SSL pipeline is structured in accordance with:
DINO (Distillation of Information via Non-parametric contrasting) (see Caron et al., “Emerging properties in self-supervised vision transformers.” In Proceedings of the IEEE/CVF International Conference on Computer Vision, pages 9650-9660, 2021, which is hereby incorporated by reference in its entirety for all purposes);
MOCO (see He, Kaiming, et al. “Momentum contrast for unsupervised visual representation learning.” Proceedings of the IEEE/CVF conference on computer vision and pattern recognition. 2020), which is hereby incorporated by reference in its entirety for all purposes);
SimCLR (see Chen, Ting, et al. “A simple framework for contrastive learning of visual representations.” International conference on machine learning. PMLR, 2020) which is hereby incorporated by reference in its entirety for all purposes).

This approach can encourage the self-supervised learning (SSL) model to capture semantically meaningful fine-grained regions of interest detailed to the pixel level. Following the scheme of unsupervised clustering, the single-class token in regular ViT is expanded to a set containing learnable multi-class tokens, assembling coarse to fine grained features to semantically aware clusters in a hierarchical manner.

SSL techniques traditionally have been used to process natural images and have not been used to process digital pathology images. Adapting existing SSL methods to histopathology data may present challenges, in that features that are important in the digital pathology context (e.g., cell density, cell morphology, co-location of dyes, etc.) are quite different than features that are extracted from natural images.

To incorporate histopathology-specific knowledge into a self-supervised contrastive learning framework, hybrid methods may be deployed. These methods may combine contrastive learning and domain-specific pretext tasks customized for histopathological patches designed according to the characteristics of the histopathological images, such as predicting magnification levels, predicting hematoxylin channel, predicting cross-stain and color normalization, etc. However, focusing on certain unique histopathological characteristics during SSL pre-training may compromise the generalizability of the model required as a universal feature extractor. For instance, a mode may selectively focus on color variances in cross-stain prediction or alternatively on the association between spatial and semantic proximity in the feature space. However, the premise assuming adjacent patches are more likely to also be adjacent at the feature level than distant patches cannot be guaranteed. As a result, noisy positive and negative pairs will jeopardize the networking training.

For example, a self-supervised multi-class-token hierarchical ViT is a novel backbone that capture both coarse and fine-grained features. (The ViT may have been tested on one or more frameworks, such as the DINO, MOCO, and/or SimCLR SSL frameworks). Compared to ImageNet pretraining and other state-of-the-art SSL methods, this model presents at least two advantages: yielding features of considerably higher quality compared to other state-of-the-art SSL frameworks and tile retrieval demonstration; learning more precise morphological phenotypes down to pixel level, different from the grid structure attention map extracted from the multi-head attention heads from regular ViT. Furthermore, the generalization gap is usually larger when an AI algorithm is trained on the data from a limited set of subjects, which may not be representative of the actual population. The disclosed SSL based paradigm can help bridge the gap by building more generalist models that learn from larger cohorts of subjects and this is possible as no manual labeling is required in the SSL. The robustness and transferability of the model are validated further in exhaustive experiments in downstream tasks such as unsupervised and semi-supervised tile classification of tissue types, and fine-grained classification of a histological feature (e.g., cytological feature).

The unique design of the backbone encoder by expanding the class tokens in regular ViT in a hierarchical manner until the final stage has potential beyond serving as a general-purpose feature extractor in two possible extensions. If trained in SSL paradigm, by equipping each histopathological image with a list of domain-specific attributes as supervisory signals for multiple auxiliary (pretext) tasks (e.g. magnification level, Hematoxylin channel), each class token at the final stage can be customized to predict each label guiding each auxiliary task simultaneously; If trained with some supervisory signals as in weakly supervised settings, each class token at the final stage can be customized to learn targeted lesion regions distinctively.

Clinical AI model requires a large amount of highly curated dataset carefully annotated by multiple medical experts driving up both the development time and costs. The present disclosure utilizes an SSL technique in the context of digital pathology. Self-supervised learning (SSL) is a form of unsupervised learning method that allows AI models to leverage unlabeled data to acquire domain-specific background knowledge to improve the performance and generalization on various downstream learning tasks. SSL is a form of unsupervised learning, designed to learn domain-specific salient features from vast amount of unlabeled data. SSL approaches can enable AI models to acquire domain-specific background knowledge from the massive amount of existing unlabeled data. It learns the visual representation based on supervised signals completely derived from the data itself. SSL can enable AI models to discover domain-specific background knowledge about the data without requiring labels from subject matter experts. That means the high-level general knowledge of the field can be learned from unlabeled data and only task-specific information or skills can be learned from the labeled data in a supervised fashion. The knowledge acquired through SSL gives the AI model an improved starting point to converge to a more robust and generalizable solution in a lesser amount through labeled training data.

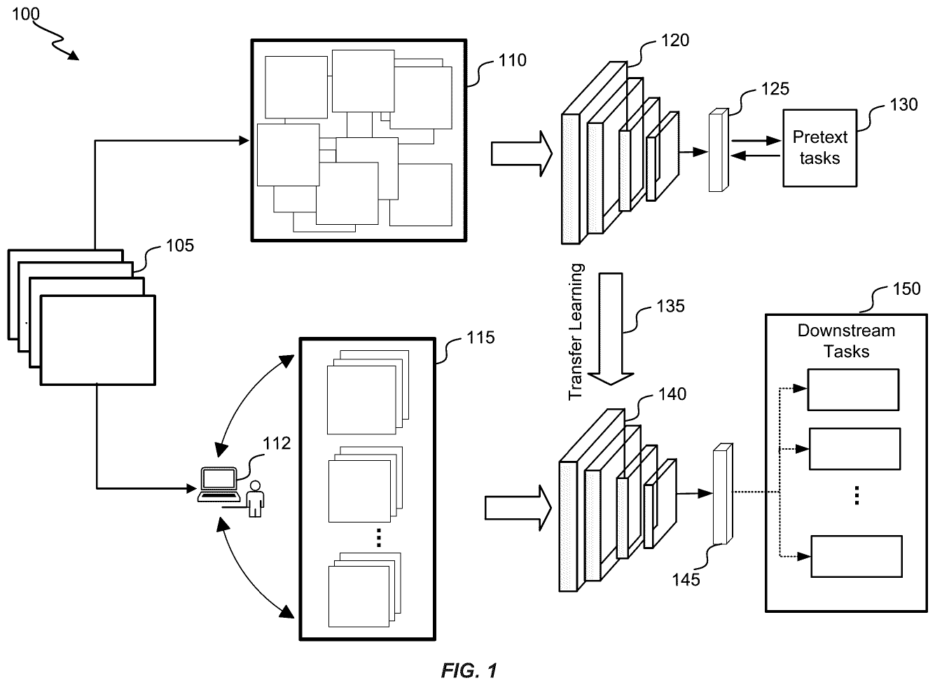

FIG. 1 illustrates an example workflow 100 of an SSL model for a given dataset 105. SSL heavily relies on unlabeled data 110 such that instead of having explicit annotations 112, a machine-learning model 120 is trained to create its own understanding of the data by generating auxiliary tasks also referred as pretext tasks 130 that are inherently related to data itself. These pretexts tasks 130 are typically designed to encourage the machine-learning model 120 to learn meaningful representations 125. For example, predicting missing part of an image, solving jigsaw puzzles, differentiating between transformed versions of the same data, predicting order of a sentence in a document or words in a sentence. Thus, acquiring domain-specific background knowledge to improve its performance and generalization on various downstream learning tasks 150. SSL implementations focus on developing domain agnostic/specific pretext tasks 130 for unlabeled data 110 to derive supervisory signals during model training. The principle of developing a pretext task 130 is to utilize the supervisory signal inferred from the unlabeled data 110 itself without depending on any external guidance. A well-learned representation 125 of the raw data is used to further facilitate the training of desired downstream tasks 150 as the initialization starter and performance improvement.

After pre-training on the SSL pretext tasks 130, the pre-trained model 120 can be fine-tuned on a smaller labeled dataset 115 for specific downstream tasks 150. This transfer learning 135 process leverages the knowledge gained during the self-supervised pre-training to improve performance on downstream tasks 150 that have limited labeled data 115. It is worth mentioning that the pre-trained machine-learning model 120 and the downstream model 140 to be utilized for downstream tasks 150 can be similar or different depending on the specific implementation and requirements. In some instances, the pre-trained model 120 can be used directly for downstream tasks 150. The idea is that the features or representations 125 learned during pre-training can also be useful to perform similar tasks. In other instances, the pre-trained model 120 can be fine-tuned for downstream tasks. Fine-tuning may involve updating the parameters of the pre-trained model 120 adapting to the specific labeled data 115 and downstream tasks 150. Alternatively, a different model can be trained for downstream tasks 150, for example, using a ViT as the pretext model 120 and a convolutional neural network (CNN) as the downstream model 140 for image classification. In this example, ViT is being used as a feature extractor and the CNN as a classifier.

Hence, in pre-training the model 120 is empowered to extract coarse and/or fine-grained features from an image dataset e.g., digital pathology images. Once the downstream model 140 is also trained, an image can be tested by first giving it to the pre-trained model 120 to extract features and then feeding into the downstream model 140 for downstream tasks (e.g., classification, captioning, segmentation etc.). To reformulate, the key to success of an SSL model may lie in wisely making use of the information derived from the image itself during pre-training.

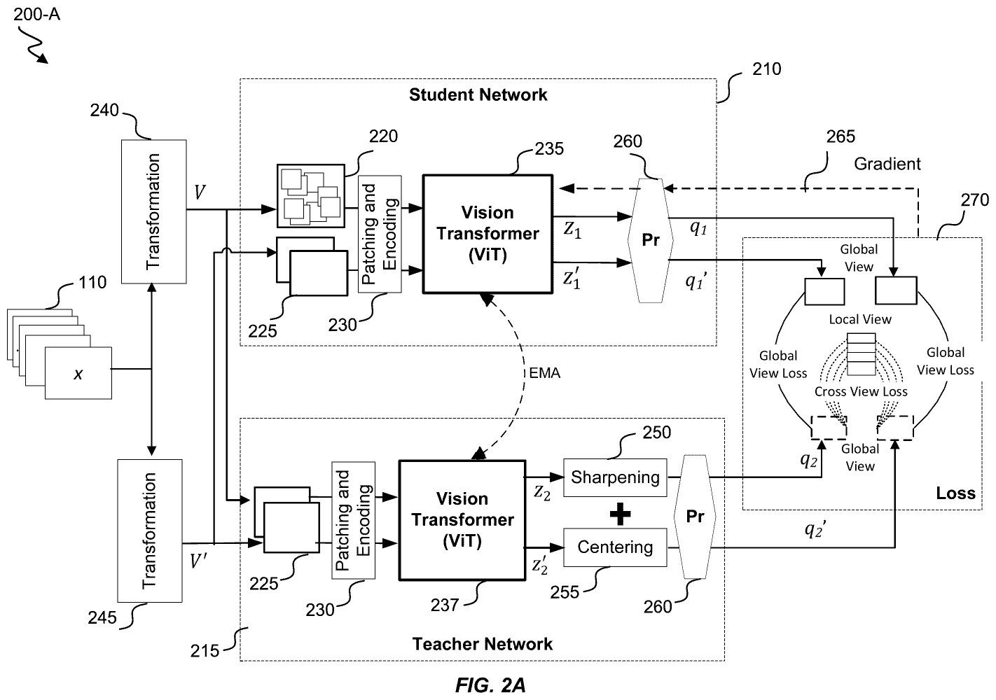

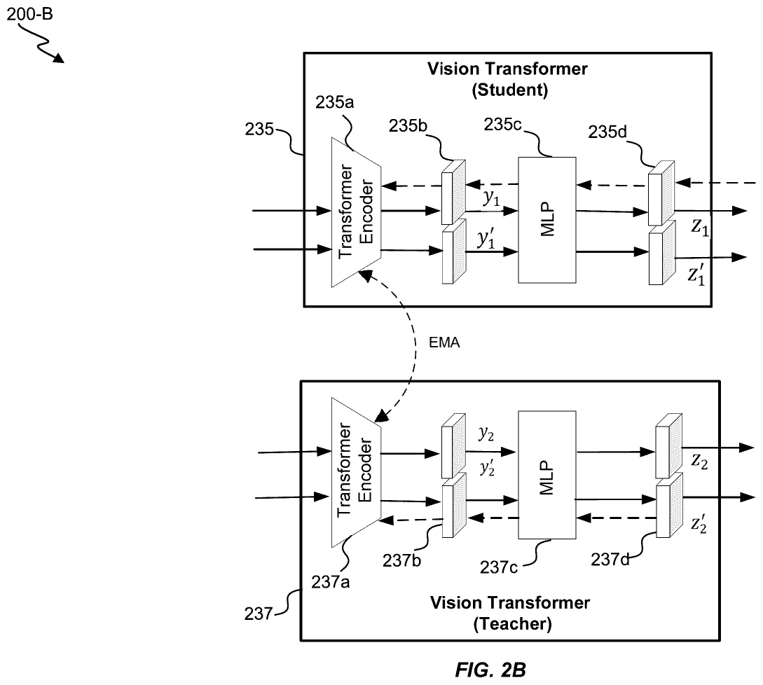

In FIG. 2A, an illustrative example of a self-supervised contrastive method, DINO (Distillation of Information via Non-parametric contrasting) is shown. The example architecture of DINO may comprise a student network 210 and a teacher network 215. These two networks student 210 and teacher 215 networks may have similar architecture but different learnable weights due to different update methods. The network 200 learns through a process called knowledge distillation in a self-supervised setting. Distillation may refer to the process of transferring knowledge from a teacher network 215 to a student network 210. The teacher-student network involves training a teacher network 215 to produce reference representations (e.g., z′₂, z₂) for the training samples 110. The student network 210, in turn, is trained to mimic the representations, and the process can be termed as knowledge distillation. The teacher 215 may be a momentum teacher, which means that the weights of the teacher network 215 are an exponentially weighted average of the weights of student network 210. DINO may use a learning objective 270 for distinguishing the representations of different augmentations of the same image using a memory bank of features from previous instances in the training data.

DINO may define a pretext task that the model needs to learn during training. The pretext task may involve augmenting the input unlabeled data 110 and training the model to distinguish between different augmentations (e.g., V 240 and V′ 245) of the input image in a self- supervised manner. For example, as depicted in FIG. 2, DINO takes an image x from the unlabeled dataset 110 and apply two different transformations or augmentations 240 and 245 to produce two different views V and V′ that are to be fed into student 210 and teacher network 215 pipelines.

A multi-crop augmentation may be applied to extract two sets of images (that may be partially overlapping) from the transformed views V and V′. Small crops may be called local views 220 (e.g., <50% of the image) and large crops (e.g., >50% of the image) may be called global views 225. In other words, the set of global views 225 are of higher dimensions than the set of local views 220. All crops are passed through the student 210 while only the global views 225 are passed through the teacher 215. This encourages “local-to-global” correspondence, training the student 210 to interpolate context from a small crop. During training, only the student 210 is trained so that the set of networks becomes able to understand that the local and global representation, although apparently different, signify the same subject. It is worth mentioning that the multi-crop augmentation and random transformations may be applied in any sequence. For example, local 220 and global views 225 can be achieved from an input image x followed by applying random augmentations (e.g., color jittering, Gaussian blur, solarization etc.) on the local 220 and global views 225 to make the network more robust.

Before feeding these views into the vision transformers (ViTs) 235 and 237, the views may be passed into patching and embedding block 230 to get the augmented embedding vectors. Patching and embedding block 230 may convert an image into equal sized patch tokens and perform a set of operations to acquire the corresponding embedding vectors for each patch. These augmented input embedding vectors to ViTs 235 and 237 may represent a sequence of embeddings of patch tokens, a learnable multi-class tokens prepended to the sequence, and the positional information.

It will be appreciated that (e.g. MOCO or SimCLR) may be used instead of or in addition to DINO.

FIG. 2B shows an illustrative example of a ViTs from FIG. 2A. The two vision transformers (ViTs) 235 and 237 in student and teacher network may have same architecture but different learnable weights due to different update method. A vision transformer (ViT) may be a type of neural network based on transformer architecture. The augmented embedded vectors from patching and embedding 230 are fed into the ViTs that may further include transformer encoders (Ees) 235a and a multi-layer perceptron (MLP) 235c in student network 210, a transformer encoder 237a (Ee,) and an MLP 237c in teacher network 215 with learnable weights θ. These transformer encoders may represent a stack of multiple self-attention layers. Self-attention may refer to a mechanism that allows the model to learn long-range dependencies between the patches for tasks such as image classification, as it may allow the model to learn how the different parts of an image may contribute to its overall label. The output of the transformer encoder is a sequence of vectors e.g., for student network 210, (y₁, y′₁) 235b representing the intermediate features of the set of global 225 and local views 220 and for teacher network 215, (y₂, y′₂) 237b representing the intermediate features of the set of global views 225.

These intermediate representations may be fed into the MLPs (235c and 237c) of the student-teacher network. The MLP (235c and 237c) in teacher-student networks may follow an MLP head 260. The MLP may act as point-wise feed-forward neural network comprising of multiple layers of linear transformations and non-linear activations. It may apply non-linear transformation to each position of the input sequence independently to produce set of projections or embeddings (z₁ and z′₁) 235d and (z₂ and z′₂) 237d for the respective student 210 and teacher networks 215. The MLP layer may help to increase the expressive power and the representation capacity of the transformer encoder.

The produced set of projections from MLP is fed to the MLP head H_{ψ}_{ₛ} 260a for student network and MLP head 260b for teacher network H_{ψ}_{ₜ} with learnable weights ψ to generate a set of probabilities (q₁, q′₁) and (q₂ and q′₂) for the respective student 210 and teacher networks 215. In the context of DINO, the MLP head may represent a component of the projection head, which is responsible for transforming the input features into a space where a learning objective 270 can be applied. The projection heads (260a and 260b) may be a layer (e.g., average pooling layer or SoftMax) or a small MLP that takes the embeddings or projections (i.e., 235d and 237d) from the respective branches as input and predicts the representations of positive pairs (augmented or transformed views of the same image) and distinguish them from negative pairs (representation from different images). The loss objective 270 aims to maximize the similarity of the two projection sets 235d and 237d from the same input while minimizing the similarity to projections of other images within the same mini batch. For contrastive metric measurement, DINO adopts cross entropy loss.

In DINO framework, mode collapse may occur during the training. There may be two forms of mode collapse: regardless of the input, the model output may always be the same along all the dimensions (i.e., same output for any input) or may be dominated by one dimension. Centering and sharpening may be deployed in teacher network 215 before the prediction head to prevent both problems. Sharpening 250 may refer to the process of refining the learned representations to make the representations more distinct and well-defined. In sharpening 250, additional operations (e.g., feature scaling, gradient clipping, temperature scaling etc.) may be applied on the projections (i.e., 235d and 237d) from the MLP 237c to enhance the features. The goal of centering 255 is to improve the clustering or concentration of similar instances in the learning feature space. Centering 255 may involve normalization, whitening, spatial attention mechanism or other techniques to improve the clustering of similar instances. Both centering 255 and sharpening 250 are performed to improve the discriminative power and clarity of the learned features in self-supervised contrastive learning process.

DINO has an asymmetric architecture for student 210 and teacher 215 network pipeline in which the weights 0 of the teacher encoder 237a (E_{θ}_{ₜ})) are updated via exponential moving average (EMA) from the student encoder 235a (E_{θ}_{ₛ}) in back propagation. The update rule is θₜ←λθₜ+(1−θₜ)θₛ with λ following a cosine schedule during training. The output probabilities from the teacher network 215, is considered as the supervisory signal to guide the training of the student network 210. The distributions of student network 210 may be matched with the teacher network 215 for the input image x by minimizing the cross-entropy loss function with respect to the parameters of the student network (i.e., θₛ, ψₛ) as given in Equation 1.

$${ℒ}_{C E} = {min}_{{θ}_{s} , {ψ}_{s}} - {H}_{{ψ}_{t}} ( {E}_{{θ}_{t}} ( x ) ) log [ {H}_{{ψ}_{s}} ( {E}_{{θ}_{s}} ( x ) ) ] \qquad Equation 1$$

The loss in Equation 1 may be adapted to self-supervised learning problem that deploys a multi-crop strategy with local 220 and global views 225 augmented from the original input image x, as in FIG. 2A. In some embodiments, the multi-crop augmentation is important but with an optimal sweet spot on the number of local views that are treated as tunable hyperparameters. The global views may be denoted as Σ_{gϵ[1,N}_{_{g}}_{]}x_{g} and several local views of smaller resolution are denoted as Σ_{lϵ[1,N}_{₁}_{]}x_{g} where Nₗ may refer to the number of local views and N_{g} may refer to the number of global views. For simplicity, only two global views are demonstrated in FIG. 2A. The loss objective function 270 as given in Equation 1 can be modified as given in Equation 2, where H_{ψ}_{ₜ} and H_{ψ}_{ₜ}, denote the output probability distributions of teacher 215 and student 210 network, respectively. The gradient propagation is stopped in teacher network 215, gradients 265 are only allowed to pass through the student network 210.

$${ℒ}_{C E} = {min}_{{θ}_{s} , {ψ}_{s}} {\sum }_{g ϵ [ 1 , {N}_{g} ]} {\sum }_{l ϵ [ 1 , {N}_{l} ]} - [ {H}_{{ψ}_{t}} ( {E}_{{θ}_{t}} ( {x}_{g} ) ) ] log [ {H}_{{ψ}_{s}} ( {E}_{{θ}_{s}} ( {x}_{l} ) ) ] \qquad Equation 2$$

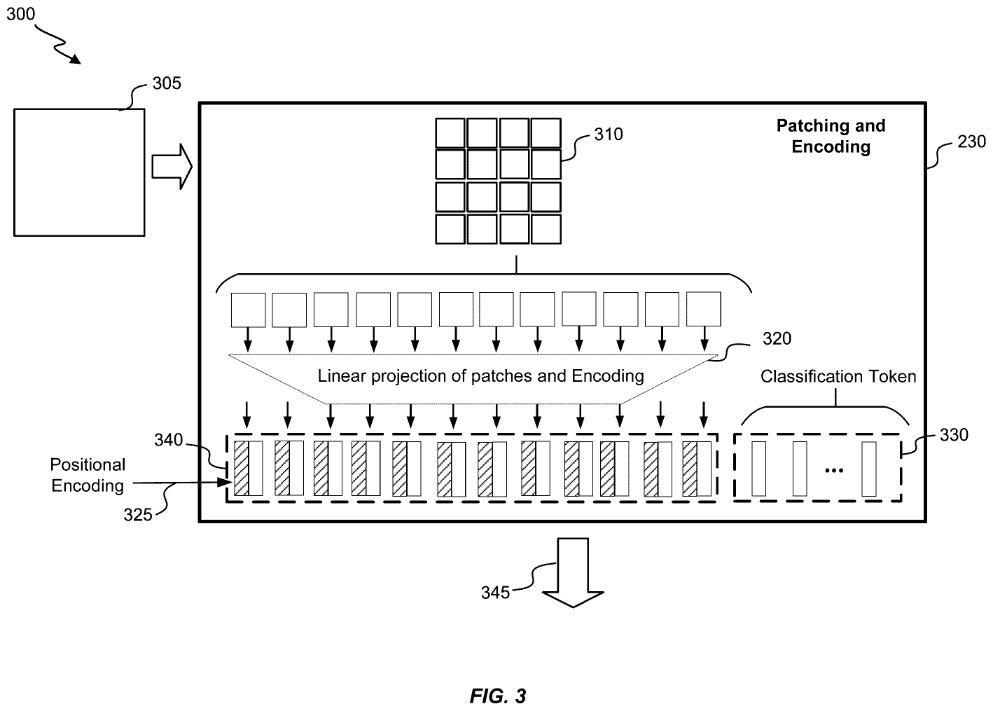

A standard transformer receives an input in one-dimensional (1D) sequence of token embeddings as it was originally designed for natural language processing (NLP). To handle two-dimensional (2D) digital pathology images, the images are reshaped into a sequence of flattened 2D patches. FIG. 3 further elaborates an example flow of the process patching and embedding 230 from FIG. 2. For structuring the input image, image 305 may be passed into the patching and embedding block 230 that may convert the image 305 into non-overlapping equal sized 2D grid of patches 310, where each patch is treated as a separate entity. Each image patch (also called token) is flattened into a 1D vector and then passed to a trainable linear projection 320 to convert the 1D high-dimensional patch into lower-dimensional vector or embedding 340. This conversion can be achieved by applying a linear transformation (e.g., fully connected layer with fewer output dimensions) on the patch embeddings. The purpose of the dimensionality reduction is to make the processing more computationally efficient while still capturing essential information from the input patches. Since vision transformers do not inherently capture the spatial information of the input, positional information needs to be incorporated. The position embeddings 325 are added to the patch embeddings 340 to retain the spatial arrangement of the patches.

In addition to position and patch embeddings 340, a special token called class [cls] token 330 may be introduced. The semantic image layout can be discovered from the attention maps of the class tokens. These attention maps may lead to promising results in unsupervised segmentation tasks. In some embodiments, unlike regular transformers, multiple class tokens 330 are used. Using a single class token may be challenging for accurate localization of different objects on a single image. Therefore, instead of a single class token multiple class tokens 330 may be used, which will be responsible for learning representations for different object classes. By doing so, the model can learn to attend to the regions of the image that belong to each class and generate class-discriminative object localization maps from the class-to-patch attentions. This technique can be useful for weakly supervised semantic segmentation, which is the task of assigning a class label to each pixel in an image using only image-level labels as supervision. The output of the linear projection block 345, a combination of patch embedding 340, positional encodings 325, and multi-class tokens 330 forms the input to the VIT.

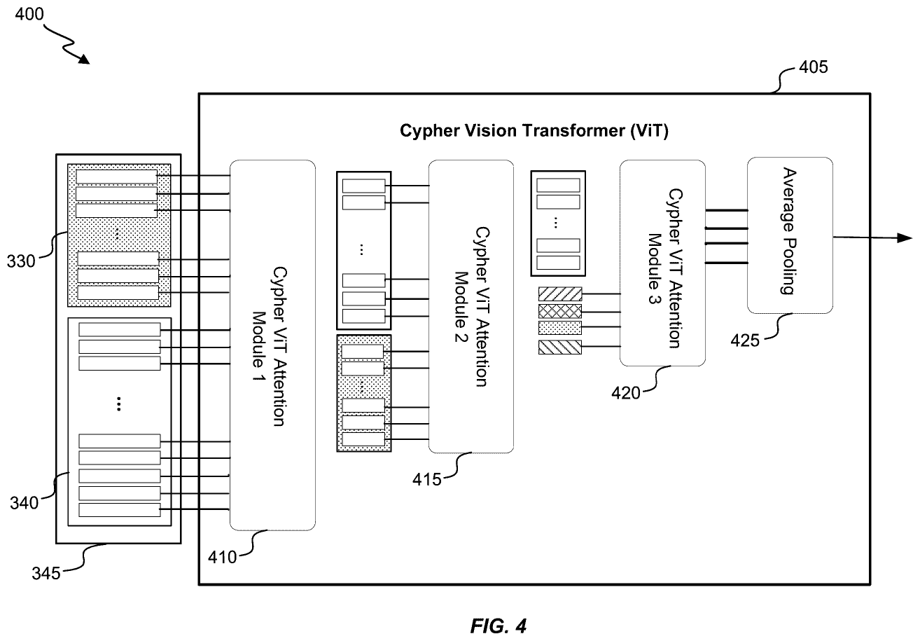

In some embodiments, SSL-based framework is provided that leverages the enormous unlabeled digital pathology data to improve the degree to which a model is used to generate digital pathology label predictions, where the model is generalizable and robust. The system may include a self-supervised backbone transformer, Cypher ViT 405 as illustrated in FIG. 4, in place of the regular ViTs (e.g., 235 and 237). Similar to ViT structure 235 expanded class tokens 330 have been included along with additional hierarchical feature agglomerative attention modules (e.g., 410, 415, 420).

As mentioned, the input image is first split into non-overlapping patches, which are then transformed into a sequence of patch tokens 340 along with positional embeddings 325. These class tokens are concatenated with patch tokens 340, embedding position information 325, to form the input tokens 345 of the transformer encoder. For the illustrative purpose, three consecutive layers of Cypher ViT attention module (i.e., 410, 415 and 420) having identical structure are shown in FIG. 4. The Cypher ViT 405 may further include an average pooling layer 425 to calculate the attention score. It may also use an MLP for classification prediction.

The goal of having class-specific tokens cannot be achieved by simply increasing the number of class tokens in ViT, because these class tokens still may not have specific meanings. To enable effective learning of high-level discriminative features of a specific object class for each class token, a class-aware training strategy for multiple class tokens 330 can be adopted. More specifically, an average pooling layer 425 can be applied on the output class tokens from the final stage of Cyber ViT attention module 420 along the embedding dimension, to generate class scores, which are directly supervised by the ground-truth class labels. Thus, a one-to-one strong connection between each class token and the corresponding class label can be built. Through this design, one significant advantage may be that the learned class-to-patch attention of different classes can be directly used as class-specific localization maps.

Regular ViT models (e.g., 235 and 237) maintain a full-length sequence in the forward pass across multiple consecutive layers of the VIT. Such a design may suffer redundancy and lack of multi-level hierarchical representations that may contribute to the successful recognition tasks in digital pathology images. One solution may be to gradually down sample the sequence length as the model goes deeper. At each stage of the Cypher VIT 405, the number of learnable class tokens 330 may be progressively decreased, driven by the intuition that as more abstract features are acquired, features can be grouped into a smaller number of clusters.

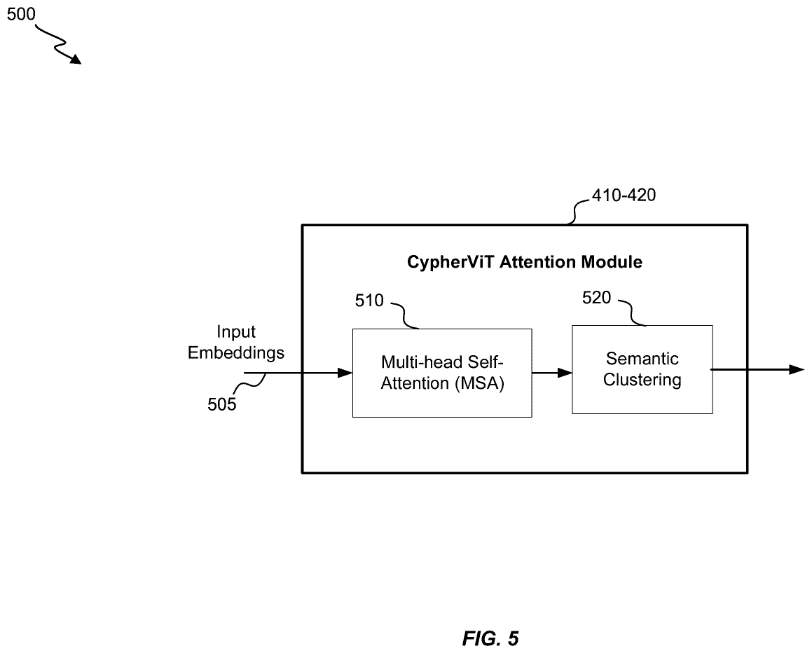

FIG. 5 illustrates an example architecture of a Cypher ViT attention module 410 from the FIG. 4. The Cypher ViT attention module 410 may include multi-head self-attention 510 and semantic clustering 520. In Cypher ViT attention module 410 the input embeddings 505 are passed to the multi-head self-attention block 510. The input embeddings 505 at each stage is different due to the hierarchical structure of the Cypher ViT 405, i.e., the output of the preceding stage becomes the input to the next stage. For example, at stage 1 410, the input embeddings 505 are from the output 345 of patching and embedding block 230, which is the concatenation of patch embedding 340, positional encoding 325 and multi-class tokens 330.

A multi-head self-attention module 510 may refer to a component of a vision transformer that may allow each input token to attend to every other token in a parallel and efficient way. The number of heads in a multi-head self-attention module 510 is a hyperparameter that can be chosen based on the task and the model architecture. Each head represents a different subspace of the input embeddings and can learn to attend to different parts of the input sequence. For each head, the query (q), key (k), and value (v) vectors having same size using linear projections are calculated by q=W_{Q}M, k=W_{K}M, v=WᵥM where M is the input embedding vector 505, and W_{q}, Wₖ, and Wᵥ are learned weighting matrices for each vector. Then, a scaled dot-product attention function can be applied as

$$Att ( q , k , v ) = SoftMax ( \frac{{qk}^{T}}{\sqrt{f}} ) v ,$$

where f may denote a scaling factor. These vectors are used to compute the relevance scores and the weighted output for each input token. The number of heads may affect the dimensionality of the query, key, and value matrices, as well as the output of the self-attention module. Typically, the number of heads is a factor of the model dimensionality to be kept e.g., 8, 12, or 16. The outputs of the different heads are then concatenated and projected to produce the final output of the module.

The multi-head attention module 510 may allow each token to attend to every other token in the sequence and produce a new feature map. The output embedding of the multi-head attention module 510 may be split as P₁ (patch tokens) and C₁ ([cls] token set) as input and apply the self-attention mechanism twice in the semantic-clustering block 520. The semantic clustering module 520 may take these patches as input and perform clustering. The output of the semantic clustering module 520 is a sequence of clustered tokens, which can be fed into the next Cypher ViT attention module (e.g., 415 or 420) or used for downstream tasks.

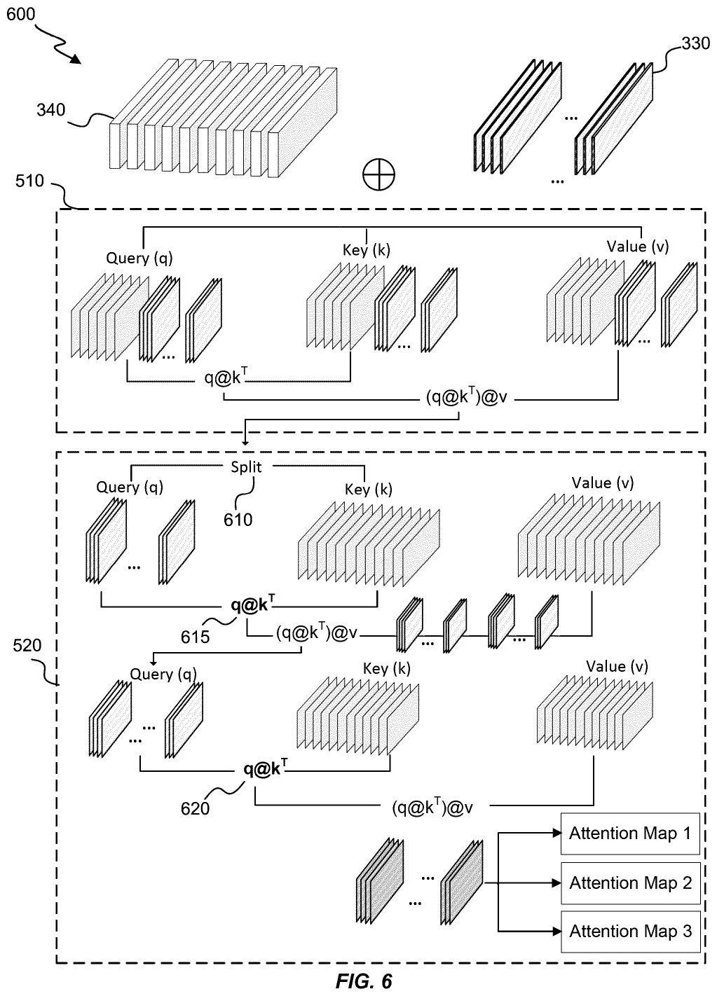

The embedded vector 345 to a multi-head-self attention module 510 may be a high-dimensional feature map that captures the global semantic information of the image. Hence, semantic clustering 520 may aim to reduce the computational complexity of self-attention in vision transformers. It may work by grouping the visual tokens that have similar semantic information into clusters, and then aggregating the key and value tokens within each cluster. This way, the number of tokens is reduced, and self-attention can be performed more efficiently. The self-attention for a single head can be reformulated for the semantic clustering 520,

$$ClustAtt ( q , k , v ) = SoftMax ( \frac{q \cdot {clust ( k ; γ )}^{T}}{\sqrt{f}} ) \cdot clust ( v ; γ ) ,$$

where a decision value γ can be computed to locate the density peaks of the cluster. Semantic clustering 520 may also preserve the global context and diversity of the original tokens, which is beneficial for visual representation learning. For clustering, semantic clustering block 520 may apply a clustering algorithm (e.g., K-means, hierarchical clustering or DBSCAN etc.) to group the pixels in the feature map into different clusters based on their similarity. Each cluster may represent a potential object category in the image. At the feature level, following a bottom-up manner without the interference of external supervisory signals, each attention module 510 aggregates patch tokens with semantically similar visual concepts into a fixed number of clusters, as demonstrated in FIG. 6.

FIG. 6 illustrates an example working of attention mechanism in accordance with an embodiment of the present disclosure. As described before, each attention module 410 in Cypher ViT may include mainly two blocks: a muti-head self-attention block 510 to explore the crosspatch relevance followed by a semantic clustering block 520 to assemble similar tokens together. The intermediate results of inherently aggregated features by the self-attention mechanism can then be preserved in the set of multiple-class tokens, defined with learnable weights during backward propagation. Then, the tokens with merged features can be fed as new input to the next stage in the hierarchical clustering pyramid as illustrated in FIG. 4. till it reaches the last stage. At final stage, each token may inherently capture a certain visual concept that corresponds to histological phenotypes such as cell, stroma, white space, etc.

To elaborate on the multi-head self-attention block 510, as illustrated in FIG. 6, the input includes two components: patch tokens 340 and class or classification [cls] token(s) 330. In FIG. 6, patch embedding 340 also includes position encoding (omitted in the figures merely for the purpose of simplifying the diagram demonstration). Patch tokens 340 remain the same as in regular ViT and are denoted as {Pᵢ}ᵢ₌₁^{N}^{_{P}}, where N_{P} refers to the number of patch tokens. However, the expansion of the [cls] to multi-[cls] tokens set-represented as {Cⱼ^{S}^{_{g}}}ⱼ₌₁^{N}^{_{c}}, where N_{c} refers to the number of learnable class tokens where s refers to stage and g refers to stage number (non-learnable fixed hyperparameter). At each stage s_{g}, the number of learnable class tokens (N_{c}) may be progressively decreased by applying clustering. Next, at the starting stage, the concatenated input of patch {Pᵢ}ᵢ₌₁^{N}_{P} and multi-[cls] tokens {Cⱼ^{S}^{ₖ}}ⱼ₌₁^{N}^{_{c}} is fed into the multi-head self-attention block 510 denoted as M^{(N}^{_{P}}^{+N}^{_{c}}^{)×d}=[{Pᵢ}ᵢ₌₁^{N}^{_{P}}, {Cⱼ^{S}^{ₖ}}ⱼ₌₁^{N}^{_{c}}], with d representing the latent space dimension. Inside the multi-head attention block 510, linear transformations are applied to generate query, key, and value at stage 1 as: q₁=W_{q1}·M^{h×(N}^{ₚ}^{+N}^{_{c}}^{)×(d/h)}, k₁=Wₖ₁·M^{h×(N}^{ₚ}^{+N}^{_{c}}^{)×(d/h)}, v₁=Wᵥ₁·M^{h×(N}^{ₚ}^{+N}^{_{c}}^{)×(d/h)}, (q₁k₁^{T})v₁=M₁^{h×(N}^{ₚ}^{+N}^{_{c}}^{)×(d/h)}, where M₁ may denote the output and M denote the input of the stage 1 of multi-head self-attention module 510, h represents the number of attention heads and each head operates on the transformed version of the input. The subscript (e.g., k₁, v₁, M₁) may represent the stage number in the hierarchy of Cypher ViT for the respective values. In FIG. 6, @ may refer to the scalar product of the two vectors.

As input for the upcoming semantic-clustering block 520,

$${M}_{1}^{h \times ( {N}_{p} + {N}_{c} ) \times ( \frac{d}{h} )}$$

may split 610 as

$${P}_{1}^{h \times {N}_{p} \times ( d / h )}$$

for patch tokens and

$${C}_{1}^{h \times {N}_{c} \times ( d / h )}$$

for the [cls] token set, and a self-attention mechanism may be applied twice as q₂=W_{q2}·C₁^{h×N}^{_{c}}^{×(d/h)}, k₂=Wₖ₂·P₁^{h×N}^{_{c}}^{×(d/h)}, v₂=Wᵥ₂·P₁^{h×N}^{_{c}}^{×(d/h)}, (q₂k₂^{T})v₂=C₂^{h×N}^{_{c}}^{×(d/h)}=C₂^{N}^{_{c}}^{×d}. Similarly, q₃=W_{q3}·C₂^{N}^{_{c}}^{×d}, k₃=Wₖ₃·P₁^{N}^{_{c}}^{×d}, v₃=Wᵥ₃·P₁^{N}^{_{c}}^{×d}, and (q₃k₃^{T})v₃=C₃^{h×N}^{_{c}}^{×(d/h)}=C₃^{N}^{_{c}}^{×d}.

C₁, C₂, and C₃ are used for equation simplification, while W_{q}, Wₖ, and Wᵥ are learnable weights in linear projections to obtain respective queries, keys, and values at each stage. The calculation of attention qk^{T} from the module above highlights the similarity between learnable multi-[cls] tokens C^{N}^{_{C}}^{×d} and P^{N}^{_{P}}^{×d}. In FIG. 6, only the attention block in the first stage is demonstrated. However, for further stages starting from the first, rather than patch and multi-[cls] tokens, the input becomes multi-[cls] tokens from the previous and current stage, i.e., the similarity matrix will be measured between C^{N}^{_{C}}^{^{s}}^{g+1}^{^{×d}} and C^{N}^{_{C}}^{^{s}}^{g×}^{^{d}}, where g>0, as shown in FIG. 4. To formulate mathematically, the attention vectors for i patch to j class at stage g (s_{g}) may be the SoftMax of the similarity matrix as given in Equation 3.

$${( q {k}^{T} )}^{{s}_{g}} = A t t {n}_{i , j}^{{s}_{g}} = \frac{exp ( {W}_{p} {P}_{i}^{{s}_{g}} \cdot {W}_{c} {C}_{j}^{{s}_{g}} + {γ}_{j} )}{{\sum }_{u = 1}^{{N}_{c}} exp ( {W}_{p} {P}_{i}^{{s}_{k}} \cdot {W}_{c} {C}_{u}^{{s}_{k}} + {γ}_{u} )} \qquad Equation 3$$

The learnable multi-[cls] token as input for the next stage can be obtained by following Equation 4, where W, W_{c}, Wₚ and W_{y} refer to the learnable weights in linear projections.

$$( q {k}^{T} ) v = {C}_{j}^{{s}_{k + 1}} = {C}_{j}^{{s}_{k}} + W \cdot \frac{{\sum }_{i = 1}^{{N}_{p}} {Attn}_{i , j}^{{s}_{k}} \cdot {W}_{v} {P}_{i}^{{s}_{k}}}{{\sum }_{i = 1}^{{N}_{p}} A t t {n}_{i , j}^{{s}_{k}}} \qquad Equation 4$$

Once the teacher-student networks are pretrained, the distilled student model, having learned from the teacher's knowledge is then fine-tuned or used directly on the downstream tasks. It is worth noting that the number of local views N₁ and number of class tokens may be unrelated in the SSL design. These two parameters can be considered as two independents tunable hyperparameters.

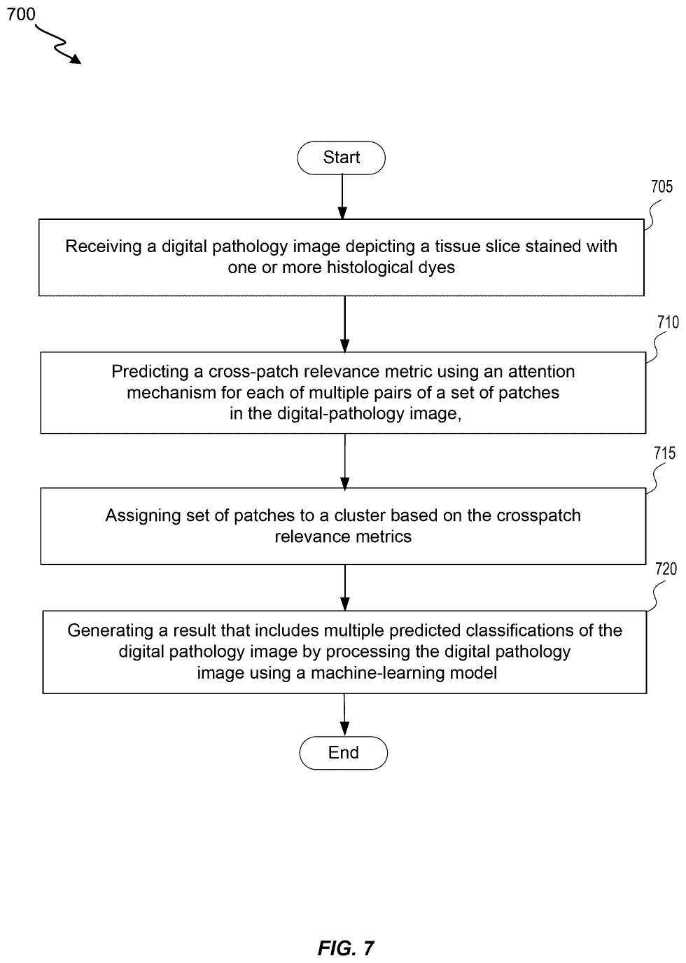

FIG. 7 shows an example flowchart of a computer-implemented method to process a digital pathology image using a machine-learning model in accordance with some embodiments of the present disclosure. At block 705, a digital pathology image is received that depicts a tissue slice stained with histological dyes and splits the image into multiple patches of equal sizes. The process at 710 relates to processing a digital pathology image using a machine learning model that includes a self-supervised hierarchical Vision Transformer (ViT) configured to perform unsupervised clustering with multiple classification tokens. The self-supervised hierarchical VIT further includes a multi-head self-attention module configured to predict a crosspatch relevance metric using an attention mechanism for each individual patch in the digital pathology image. At block 715, the individual patches are assigned to a cluster based on the crosspatch relevance metrics. Finally, at block 720, a result is generated that includes multiple predicted classifications of the digital pathology image by the machine-learning model utilizing Cypher ViT.

### Example Implementation

An example implementation of the framework is provided for Cluster-based histopathology Phenotype Representation learning by self-supervised multi-class-token hierarchical ViT (CypherViT) 405 (as illustrated in FIG. 4) as the novel backbone encoder to replace the regular ViT 235 in the SSL pipeline inherited from DINO. (It will be appreciated that various other encoders may be used, such as one inherited from MOCO or SimCLR) This approach proves to capture semantically meaningful fine-grained regions of interest detailed to the pixel level. Following the scheme of unsupervised clustering, the single-class token in regular ViT is expanded to a set containing learnable multi-class tokens 330, assembling coarse to fine grained features to semantically aware clusters in a hierarchical manner.

In the following demonstration, all models were pre-trained without using any labels. Processing was distributed on 4 GPUs that were connected in parallel. The batch size was 100 per GPU with AdamW as the optimizer for the student network. For patch embedding, the patch size is set as 16. Following the implementation setting in DINO, the base learning rate 5e-04 and batch size of 256 was configured to be linearly scaling up during the first 10 epochs and then decayed with a cosine schedule. The weight decay followed the same schedule from 0.04 to 0.4. To provide different views as demonstrated in FIG. 2, the data augmentation design used in DINO was adopted. To elaborate, the augmentation pipeline for creating a global view included random resize crop, random horizontal flip, color jittering, gaussian blur, solarization, and normalization. For local views, the multi-crop strategy was adopted to randomly crop the input image to half its original size. From experiments, it was determined that adding the right amount number of local views helps stabilize the SSL training and boost the performance.

### VGH Dataset

The network is trained on VGH (Vancouver General Hospital) dataset, which is the hematoxylin and eosin (H&E) breast cancer dataset built from the Netherlands Cancer Institute (NKI) cohort and the VGH cohort. Patches with less than 70% tissue coverage were filtered out. Patches were cropped to smaller sizes of 224×224 pixels from the original resolution of 1128×720 pixels, with up to 50% overlap. The dataset is also augmented by applying transformations 240 involving rotations of 90, and 180 degrees, and vertical and horizontal inversion. In total, the post-processed dataset contains ≈300,000 images.

### BreastPathQ Dataset

BreastPathQ is a challenging dataset with noisy and fine-grained labels. For training/validation, a set of 2,579/187 patches were extracted from 96 H&E slides at 20 times magnification with residual invasive breast cancer from the TCGA-BRCA cohort measuring tumor cellularity i.e., the fractional occupancy of tumor cell presence in the image patch. Each patch has been assigned a tumor cellularity score on a continuous scale from 0 to 1. Accordingly, mean-squared error (MSE) has been reported in TABLE 1 using linear regression and Kendall- Tau concordance. The Kendall tau correlation coefficient is a non-parametric measure of association based on the number of concordances and discordances in paired observations.

### PanNuke Dataset

PanNuke dataset comprises semi-automatically generated nuclei instance images with exhaustive nuclei labels across 19 different tissue types sampled from more than 20,000 whole slide images at different magnifications, from multiple data sources. In total the dataset includes 205,343 labeled nuclei, each with an instance segmentation mask. However, the labels are used only for experimentation purposes.

### CRC Dataset—Downstream Taks (Patch level Tissue Phenotyping)

As training set in downstream tasks 150, CRC (colorectal cancer) both with and without Macenko normalization) include 100,000 hematoxylins & cosin (H&E) stained 224×224 histological patches at 20 times magnification of human colorectal cancer (CRC) and normal tissue manually extracted from 86 slides. Each image is annotated with a type of tissue label (adipose (Adi), background (Back), debris (Deb), lymphocytes (Lym), mucus (Muc), smooth muscle (Mus), normal colon mucosa (Norm), cancer-associated stroma (Str), colorectal adenocarcinoma epithelium (Tum)).

For evaluation, standard protocols are employed on four (above-mentioned) datasets by either using frozen features or fine-tuning the features. The k-nearest neighbor (k-NN) classifier and a linear classifier (linear probing) are trained on frozen features extracted from a pre-trained SSL backbone by sweeping over different numbers of nearest neighbors for KNN and different learning rates for linear probing. Furthermore, networks were initialized with the pre-trained weights to conduct a semi-supervised experiment using different percentages of annotated images evenly distributed to each class in the training set, while the testing data remains the same as the official splitting.

The results of KNN accuracy and linear probing accuracy on patch-level tissue type classification evaluated on CRC dataset with and without normalization and PanNuke dataset are reported in TABLE 1. Moreover, TABLE 1 also shows mean square error (MSE) and Kendall-Tau concordance score on BreastPathQ dataset. The best results among different hyper-parameter settings (as shon in TABLE 4) are reported for the DINO Cypher ViT model. All SSL methods are using the vanilla ViT backbone except DINO CypherViT, which uses the proposed CypherViT backbone. The arrows next to the labels in all the tables below refer to the indication of direction in which the value of the respective metric should be e.g., an up arrow (↑) suggest the more the better and a down arrow (↓) suggests the lesser the better. The results in the tables for the present disclosure are bold for highlighting purpose. In TABLE 1, Acc @1 metric (also referred as Top-1) may suggest the percentage of instances where correct label is the top prediction made by the model. Similarly, Acc @3 may suggest whether the correct label is among the top three predictions made by the model. It is relatively lenient metric than Acc @1.

TABLE 1
Accuracies of KNN and linear probing on patch-level tissue type classification evaluated
on CRC dataset (with and without normalization), PanNuke and BreastPathQ dataset.

| | CRC (without normalization) | | | | CRC (with normalization) | | | | PanNuke | | |
|---|---|---|---|---|---|---|---|---|---|---|---|
| | KNN | | Linear probing | | KNN | | Linear probing | | KNN | BreastPathQ | |
| Method | Acc@1↑ | Acc@3↑ | Acc@1↑ | Acc@3↑ | Acc@1↑ | Acc@3↑ | Acc@1↑ | Acc@1↑ | Acc@1 | MSE↓ | Tau↑ |
| Pretrained | 77.99 | 86.51 | 85.50 | 98.18 | 82.29 | 88.89 | 87.03 | 98.41 | 79.78 | 0.126 | 0.357 |
| on ImageNet | | | | | | | | | | | |
| imCLR | 80.44 | 94.03 | 85.65 | 98.28 | 88.25 | 96.83 | 88.77 | 98.83 | 82.86 | 0.049 | 0.510 |
| MOCO | 83.73 | 95.23 | 85.96 | 97.91 | 84.51 | 94.65 | 87.77 | 99.22 | 81.36 | 0.198 | 0.278 |
| DINO | 84.01 | 95.41 | 86.31 | 98.77 | 90.38 | 96.84 | 91.42 | 99.28 | 90.09 | 0.031 | 0.620 |
| iBOT | 84.57 | 94.30 | 87.42 | 98.97 | 91.48 | 94.90 | 92.67 | 99.60 | 89.46 | 0.038 | 0.608 |
| CypherViT | 86.53 | 98.27 | 90.67 | 99.04 | 93.21 | 98.96 | 94.47 | 99.61 | 93.67 | 0.021 | 0.690 |

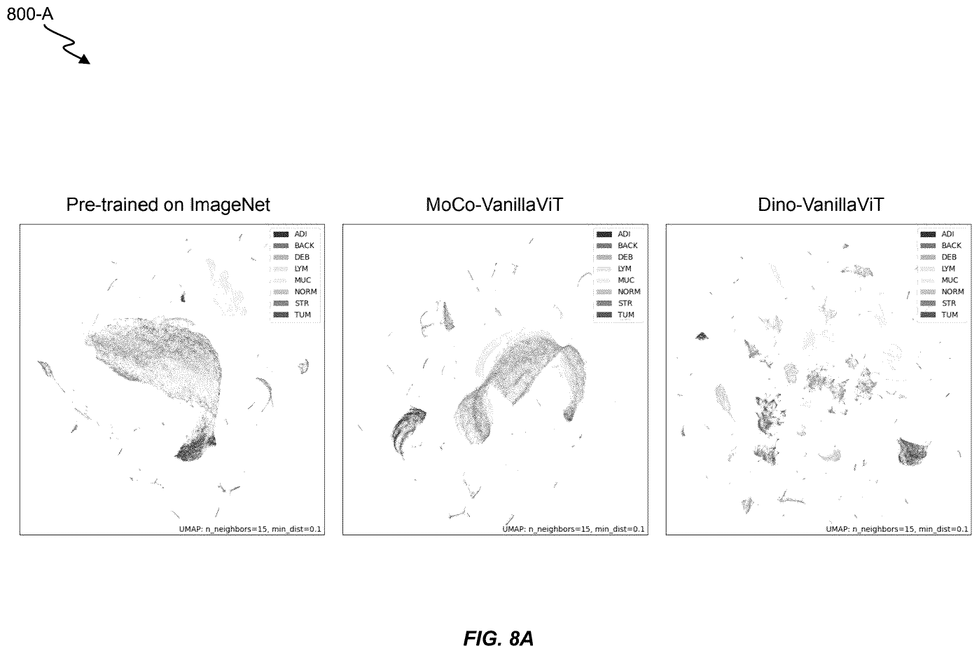

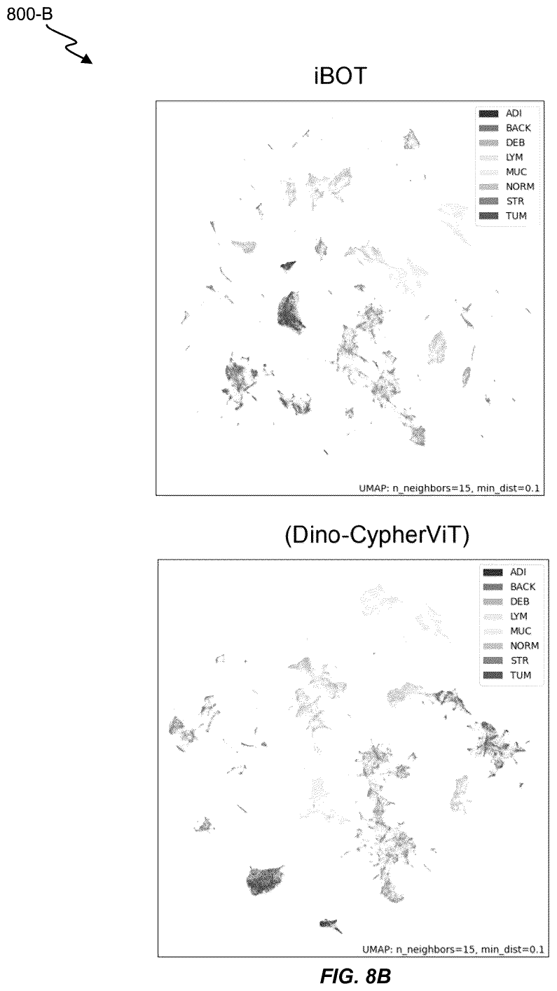

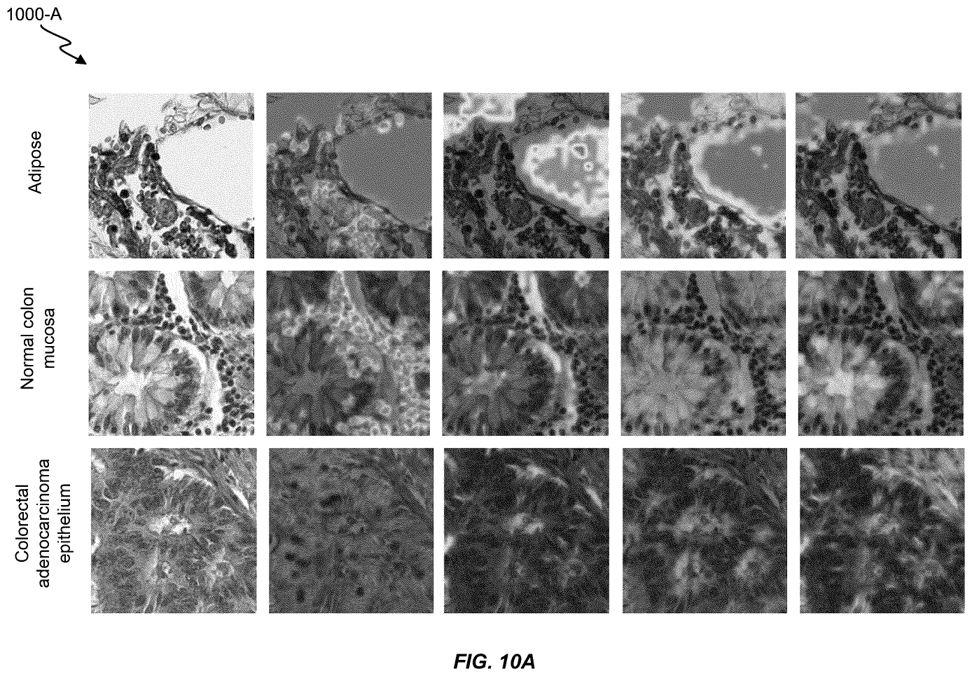

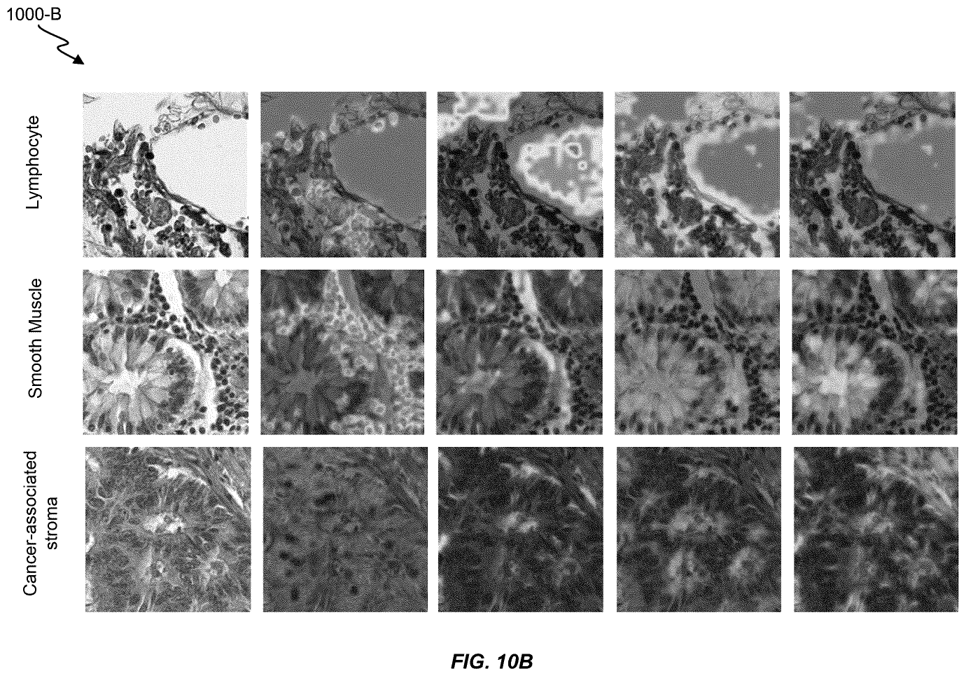

In TABLE 2, the performance variations of the disclosed Cypher ViT model with DINO framework trained with various percentages (e.g., 1%, 5%, 10%, 20%, 50%, and 100%) of labeled CRC dataset with Macenko normalization are examined. For fair comparison, other existing state-of-the-art self-supervised methods trained on the VGH dataset are also implemented with the same architecture (ViT-small) following the default hyper-parameters setting in the officially released codebases. All SSL methods are using the Vanilla ViT backbone except DINO-CypherViT which uses the proposed CypherViT backbone. Addition of 5% labeled data can provide comparable results to the performance using the entire training dataset, which is surpassed when increasing labeled data to 10%. It suggests promising SSL applications to achieve comparable or even better performance via training on much fewer data. The evaluation results and ablation studies are shown in FIG. 8A, FIG. 8B, FIG. 10A and FIG. 10B.

TABLE 2
Semi-supervised learning accuracy on patch-level tissue type
classification evaluated with different percentages of labeled
CRC dataset (with normalization) used in training.

| | CRC (With Normalization) | | | | | |
|---|---|---|---|---|---|---|
| Method | 1% | 5% | 10% | 20% | 50% | 100% |
| Fully supervised | 51.64 | 74.04 | 86.50 | 88.94 | 90.66 | 91.87 |
| MoCo | 73.32 | 88.30 | 90.18 | 90.19 | 91.07 | 92.05 |
| iBOT | 73.36 | 90.56 | 91.91 | 92.20 | 92.34 | 92.84 |
| DINO | 72.42 | 89.61 | 92.05 | 92.20 | 93.48 | 93.76 |
| DINO Cypher ViT | 76.36 | 91.10 | 93.11 | 93.20 | 93.98 | 94.82 |

TABLE 3 shows the top-1 KNN classification accuracy of the disclosed CypherViT 405 based DINO framework with 9 tissue types performing downstream tasks with labeled CRC dataset with normalization. For compact illustration the complete names of tissues have been replaced with the acronyms as follows: adipose (Adi), background (Back), debris (Deb), lymphocytes (Lym), mucus (Muc), smooth muscle (Mus), normal colon mucosa (Norm), cancer- associated stroma (Str), colorectal adenocarcinoma epithelium (Tum)).

TABLE 3
Top-1 KNN accuracy of 9 tissue types from CRC dataset with normalization.

| | CRC (With Normalization) | | | | | | | | |
|---|---|---|---|---|---|---|---|---|---|
| Method | ADI | BACK | DEB | LYM | MUC | MUS | NORM | STR | TUM |
| Fully supervised | 89.28 | 93.59 | 92.60 | 80.28 | 82.36 | 71.42 | 85.91 | 65.63 | 78.09 |
| MOCO | 90.17 | 95.26 | 94.36 | 85.78 | 84.90 | 78.23 | 87.01 | 65.91 | 81.90 |
| SimCLR | 95.69 | 99.74 | 96.47 | 93.13 | 88.43 | 83.70 | 89.51 | 66.47 | 87.58 |
| DINO | 97.18 | 99.87 | 97.18 | 94.54 | 92.55 | 85.51 | 89.20 | 67.88 | 90.13 |
| iBOT | 97.26 | 98.07 | 98.59 | 97.00 | 92.45 | 88.93 | 88.73 | 68.16 | 90.87 |
| CypherViT | 98.90 | 100.0 | 99.64 | 98.23 | 93.46 | 92.54 | 91.29 | 72.26 | 92.61 |

(Note:
adipose (Adi), background (Back), debris (Deb), lymphocytes (Lym), mucus (Muc), smooth muscle (Mus), normal colon mucosa (Norm), cancer-associated stroma (Str), colorectal adenocarcinoma epithelium (Tum))

FIG. 8A illustrates 2D UMAP (uniform manifold approximation and projection) visualization of feature embeddings extracted from an example implementation of models pre-trained on ImageNet, existing state-of the-art self-supervised learning (SSL) frameworks MoCo (Momentum Contrast) and DINO pre-trained on VGH dataset. FIG. 8B illustrates 2D UMAP visualization of feature embeddings extracted from an example implementation of a state-of-the-art SSL framework iBOT (image BERT training with Online Tokenizer) and the present disclosure pre-trained on VGH dataset. UMAP is a dimensionality reduction technique known for preserving global and local structures in the data.

The existing state-of-the-art SSL models (MoCo, DINO, iBOT), and the model in accordance with an embodiment of the present disclosure are pretrained on VGH dataset. Default parameters setting for UMAP plotting is kept as (neighbors=15, dist=0.1). To investigate results from stage 3, the extracted feature

$${C}_{3}^{{N}_{C}^{{s}_{k} \times d}}$$

is estimated using interpolation, and the attention map is visualized by overlaying it on the original image, as shown in FIG. 8. The feature embeddings are obtained by employing SSL backbone encoders on the CRC dataset with normalization for downstream tasks. For the loss objective calculation, an average pooling layer was applied to aggregate outputs from the semantic-clustering block 520 at the final stage. It can be observed from FIG. 8A and 8B, feature embeddings from DINO with the proposed CypherViT backbone present cleaner and much less noisy clustering results than other state-of- the-art SSL approaches. CypherViT 405 based DINO framework performs better in terms of concentration and intra and inter-clustering. Interestingly, the post-clustering features extracted from learnable multi-[cls] tokens 330 manifest interpretable attention-focused regions that correspond to morphological tissue phenotypes of histopathological patches.

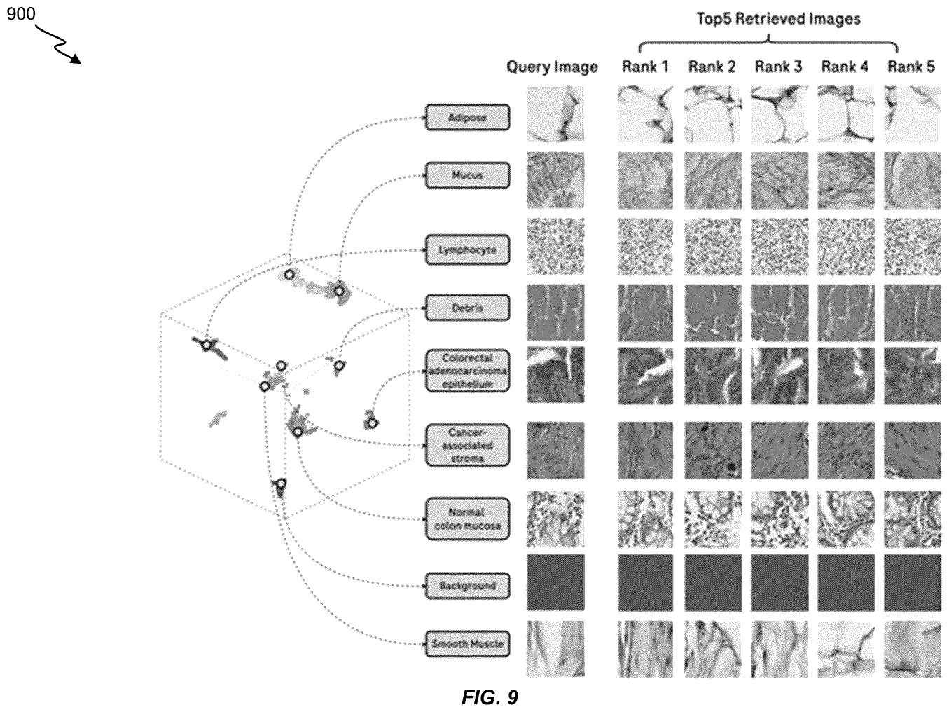

FIG. 9 shows retrieval results from a set of query images in accordance with an example implementation of the present disclosure. The top five retrieved images of each query image are shown in each row for 9 tissue types from CRC dataset. A database of embeddings for each patch is created distributed discriminately in embedding space. When a query image is selected, its embedding is compared with those in the database based on cosine distance. Following this process, L most similar patches are returned, where L can be customized.

In FIG. 10, attention maps for six tissue types from the CRC dataset with normalization from an example implementation of the disclosed Cypher ViT 405 integrated as a backbone network within the SSL-based DINO framework. The best results on relatively high attention scores representing precise identification of morphological phenotypes at the pixel level are highlighted in the first column of FIG. 10A and 10B.

FIG. 10A shows attention maps of three types of predicted classification tokens (i.e., Adipose, normal colon mucosa and colorectal adenocarcinoma epithelium) extracted from the learnable multi-class tokens 330 at a final stage of the example implementation of the present disclosure. FIG. 10B shows attention maps of other three predicted classification tokens (i.e., lymphocyte, smooth muscle, cancer-associated stroma) extracted from the learnable multi-class tokens at the final stage of the example implementation of the present disclosure. FIG. 10A and 10B depict precise identification of morphological phenotypes at the pixel level, surpassing the grid structure attention map derived from multi-head attention 510 in CypherViT 405. It can also be observed that the semantic clustering block can learn semantically interpretable features corresponding to distinct morphological phenotypes.

Semantically meaningful attention maps from multi-[cls] tokens suggest promising interpretability. To provide a more intuitive visualization of what CypherViT 405 has learned from SSL training, the original image with attention maps is overlayed after interpolating the attention weights extracted from each [cls] token at the final stage of semantic clustering blocks 520. It can be observed in FIG. 10A and 10B that the regions of interest highlighted from learned attentions indicate morphological phenotypes, such as cell in the first column, white space in the second column, and stroma tissue in the last two columns in each class shown. Compared to state-of-the-art self-supervised ViT models such as DINO, Cypher ViT has two advantages regarding interpretability. Firstly, the attention map presents more fine-grained and precise details in contrast to previous SSL models that provide attention maps in grid structure constrained within regular shape; secondly, the disclosed design has more developmental potential to accommodate other machine learning settings for future work. To elaborate, similar to unsupervised clustering, although what each [cls] token actually learns is unknown, it can, however, be easily tweaked in a weakly-supervised experiment setting by slight modification on the loss to each [cls] token to control what each [cls] token should focus on based on what is assigned.

The comparison TABLE 4 presents the results of an ablation study that investigates two SSL families: generative SSL (including MAE) and contrastive SSL using the same SSL framework (including SimCLR, MoCo, and Dino). The SSL frameworks are implemented with two different backbones: Vanilla ViT and the disclosed CypherViT. TABLE 4 reports the KNN accuracy for patch-level tissue type classification, evaluated on the CRC dataset with normalization and the PanNuke dataset. Additionally, it includes the MSE and Kendall-Tau concordance score for the BreastPathQ dataset. As observed, it is evident that the proposed CypherViT 405 is a plug-and-play solution that can seamlessly integrate into different SSL frameworks. It demonstrates excellent performance and is model agnostic, making it adaptable to various scenarios.

TABLE 4
A comparison table investigating two SSL families, generative and
contrastive using the same SSL framework with two different
backbones: Vanilla ViT and the proposed Cypher ViT.

| | CRC | | | |
|---|---|---|---|---|
| | (With Normalization) | PanNuke | BreastPathQ | |
| Method | Accuracy↑ | | MSE↓ | Tau↑ |
| MAE | 73.83 | 74.78 | 0.265 | 0.211 |
| SimCLR | | | | |
| Vanilla ViT | 88.25 | 82.86 | 0.049 | 0.510 |
| Cypher ViT | 88.99 | 84.75 | 0.027 | 0.638 |
| MoCo | | | | |
| Vanilla ViT | 84.51 | 81.36 | 0.198 | 0.278 |
| Cypher ViT | 86.94 | 83.37 | 0.079 | 0.531 |
| DINO | | | | |
| Vanilla ViT | 90.38 | 89.46 | 0.038 | 0.608 |
| Cypher ViT | 93.37 | 93.67 | 0.021 | 0.690 |

Moreover, the disclosed CypherViT 405 is an SSL framework agnostic backbone that seamlessly integrates into different contrastive-based SSL pipelines like DINO, MoCo, and SimCLR. Comprehensive experiments have been conducted to demonstrate its consistent performance improvement compared to the vanilla ViT backbone, as shown in TABLE 4. Its “plug-and-play” capability allows for easy and efficient framework adaptation without significant architectural modifications.

Features may be more robust with proper number of local views from multi-crop augmentation depending on tasks. To study the contribution of key hyper-parameters used in the main architecture of CypherViT 405 and significant technique to stabilize training, ablation studies in TABLE 5 is conducted, testing performance variations from different combinations of [cls] token number at the final stage and number of local views. It can be inferred that more local views are beneficial for tasks requiring learned features on a more fine-grained level, such as the calculation of MSE error between linear regression outcome with annotations from BreastPathQ that measures tumor cellularity. While on coarse-grain level, or global features for classification tasks, fewer local views are preferred. For the experiments carried out in TABLE 5, using four [cls] tokens at final stage and two local views from muti-crop augmentation appears to be the optimal hyper-parameter combination.

TABLE 5
An ablation study on two components of the proposed DINO-CypherViT, the
number of [cls] tokens for feature clustering used at the last stage,
and the number of local views used in the multi-crop augmentation strategy.

| | | CRC (Without | | CRC (With | | | | | |
|---|---|---|---|---|---|---|---|---|---|
| Ablations | | Normalization) | | Normalization) | | PanNuke | | | |
| # of | # of | Accuracy | | | | | | BreastPathQ | |
| [cls] | Local Views | @1 | @3 | @1 | @3 | @1 | @3 | MSE↓ | Tau↑ |
| 4 | 0 | 85.80 | 96.50 | 91.74 | 97.24 | 91.75 | 97.13 | 0.029 | 0.65 |
| 4 | 2 | 87.38 | 98.06 | 93.37 | 99.26 | 93.67 | 96.88 | 0.026 | 0.66 |
| 4 | 4 | 89.05 | 98.90 | 92.92 | 99.13 | 93.15 | 96.80 | 0.021 | 0.69 |
| 4 | 8 | 87.70 | 97.10 | 92.33 | 97.98 | 92.28 | 96.95 | 0.023 | 0.69 |
| 8 | 0 | 85.36 | 97.03 | 88.50 | 98.44 | 87.65 | 95.70 | 0.028 | 0.64 |
| 8 | 2 | 85.48 | 98.41 | 93.23 | 98.96 | 91.75 | 96.27 | 0.028 | 0.63 |
| 8 | 4 | 86.53 | 98.27 | 91.50 | 98.19 | 90.51 | 96.15 | 0.025 | 0.66 |
| 8 | 8 | 86.14 | 97.57 | 90.58 | 97.38 | 89.87 | 95.48 | 0.022 | 0.70 |

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

The present description provides preferred exemplary embodiments only, and is not intended to limit the scope, applicability or configuration of the disclosure. Rather, the present description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the present description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

What is claimed is:

1. A computer-implemented method comprising:
receiving a digital pathology image that depicts a tissue slice stained with one or more histological dyes;
generating a result that includes multiple predicted classifications of at least part of the digital pathology image by processing the digital pathology image using a machine-learning model, wherein the machine-learning model includes a self-supervised hierarchical Vision Transformer (ViT) configured to perform unsupervised clustering with multiple classification tokens; and
outputting the result.

2. The computer-implemented method of claim 1, wherein the multiple predicted classifications include a type of tissue.

3. The computer-implemented method of claim 1, wherein the multiple predicted classifications include a magnification level.

4. The computer-implemented method of claim 1, wherein the multiple predicted classifications include a diagnostic category characterizing a histological feature.

5. The computer-implemented method of claim 4, wherein the diagnostic category includes a nondiagnostic category, negative for malignancy category, atypical category, neoplastic: benign category, suspicious category, or positive for malignancy category.

6. The computer-implemented method of claim 1, wherein the multiple predicted classifications include a category predicting whether the digital pathology image depicts a particular histological feature of malignancy or an extent to which the digital pathology image depicts the particular histological feature of malignancy.

7. The computer-implemented method of claim 6, wherein the particular histological feature of malignancy includes: high cellularity, cellular enlargement, cellular discohesiveness, a high nuclear-to-cytoplasm ratio, nuclear hyperchromasia, prominent nucleoli, large nucleoli, abnormal nuclear-chromatin distribution, high mitotic activity, abnormal nuclear membrane, cellular pleomorphism, nuclear pleomorphism, or tumor diathesis.

8. The computer-implemented method of claim 1, wherein the multiple predicted classifications include, for each of a set of portions of the digital pathology image, a classification characterizing what is being depicted within the portion.

9. A system comprising:
one or more data processors; and
a non-transitory computer readable storage medium containing instructions which, when executed on the one or more data processors, cause the one or more data processors to perform actions including:
receiving a digital pathology image that depicts a tissue slice stained with one or more histological dyes;
generating a result that includes multiple predicted classifications of at least part of the digital pathology image by processing the digital pathology image using a machine-learning model, wherein the machine-learning model includes a self-supervised hierarchical Vision Transformer (ViT) configured to perform unsupervised clustering with multiple classification tokens; and
outputting the result.

10. A computer-program product tangibly embodied in a non-transitory machine- readable storage medium, including instructions configured to cause one or more data processors to perform action including:
receiving a digital pathology image that depicts a tissue slice stained with one or more histological dyes;
generating a result that includes multiple predicted classifications of at least part of the digital pathology image by processing the digital pathology image using a machine-learning model, wherein the machine-learning model includes a self-supervised hierarchical Vision Transformer (ViT) configured to perform unsupervised clustering with multiple classification tokens; and
outputting the result.

11. The computer-program product of claim 10, wherein the multiple predicted classifications include a type of tissue.

12. The computer-program product of claim 10, wherein the multiple predicted classifications include a magnification level.

13. The computer-program product of claim 10, wherein the multiple predicted classifications include a diagnostic category characterizing a histological feature.

14. The computer-program product of claim 13, wherein the diagnostic category includes a nondiagnostic category, negative for malignancy category, atypical category, neoplastic: benign category, suspicious category, or positive for malignancy category.

15. The computer-program product of claim 10, wherein the multiple predicted classifications include a category predicting whether the digital pathology image depicts a particular histological feature of malignancy or an extent to which the digital pathology image depicts the particular histological feature of malignancy.

16. The computer-program product of claim 15, wherein the particular histological feature of malignancy includes: high cellularity, cellular enlargement, cellular discohesiveness, a high nuclear-to-cytoplasm ratio, nuclear hyperchromasia, prominent nucleoli, large nucleoli, abnormal nuclear-chromatin distribution, high mitotic activity, abnormal nuclear membrane, cellular pleomorphism, nuclear pleomorphism, or tumor diathesis.

17. The computer-program product of claim 10, wherein the multiple predicted classifications include, for each of a set of portions of the digital pathology image, a classification characterizing what is being depicted within the portion.

18. The computer-program product of claim 17, wherein each of the set of portions is a patch.

19. The computer-program product of claim 17, wherein each of the set of portions is a pixel.

20. The computer-program product of claim 10, wherein the self-supervised hierarchical Vision Transformer (ViT) includes a multi-head self-attention module configured to:
predict, for each of multiple pairs of a set of patches in the digital pathology image, a crosspatch relevance metric using an attention mechanism; and
assign each of one or more of the set of patches to a cluster based on the crosspatch relevance metric.
